(19) United States
(12) Patent Application Publication
(10) Pub. No.: US 2015/0133817 A1
(43) Pub. Date: May 14, 2015

(54) PEDIATRIC SALIVA SAMPLE COLLECTION SYSTEM

(71) Applicant: OASIS DIAGNOSTICS CORPORATION, VANCOUVER, WA (US)

(72) Inventors: CHRISTOPHER C. SLOWEY, VANCOUVER, WA (US); PAUL D. SLOWEY, VANCOUVER, WA (US); ROBERT L. BUCK, FAIRVIEW, OR (US)

(21) Appl. No.: 14/537,763

(22) Filed: Nov. 10, 2014

Related U.S. Application Data
(60) Provisional application No. 61/901,961, filed on Nov. 8, 2013.

(51) Int. Cl. A61B 10/00

(52) U.S. Cl. CPC A61B 10/0051; A61J 2017/001
USPC 600/582

(57) ABSTRACT

A pediatric saliva collection system includes a pacifier portion, a stiffener, a collector, and a pad compression tube. The pacifier portion includes a receiver having a first interior cavity and nipple having a second interior cavity in fluid communication with the first interior cavity, the nipple including a plurality of apertures. The stiffener is insertable into the pacifier receiver, and adapted to receive the collector therein, the collector including a handle, an absorbent pad extending out from the handle and a seal; the collector removably insertable into the pacifier portion; and, the pad compression tube insertable over the collector to compress the absorbent pad and express liquid sample.

### CROSS-REFERENCE TO OTHER APPLICATIONS

This application is a nonprovisional of, and claims priority to, co-pending U.S. Provisional Patent Application Ser. No. 61/901,961, filed Nov. 8, 2013, which is hereby incorporated by reference into this application.

### FIELD OF THE INVENTION

This application is directed to apparatus and methods for obtaining saliva samples from infants, including premature infants.

### BACKGROUND

Using saliva samples rather than blood sampling for diagnosis for neonatal and pediatric patients would be very desirable in order to reduce stress and trauma to these vulnerable patients. Advancements in using saliva to diagnose many conditions previously requiring blood samples makes this plausible, but apparatus designed to collect saliva samples from adults may not be usable for such small patients—both due to the smaller sizes of their mouths, their inability to comply with instructions, and the smaller volumes of saliva collectable from these small patients, especially in neonatal care facilities. Additionally, significant safety concerns arise, as the collector must be positively captured to prevent any possibility of detaching in the mouth and creating a choke hazard. Additionally, all parts must be designed to prevent damage to the oral cavities.

An additional difficulty with pediatric saliva collection is providing an ability to incorporate sample adequacy indication, due to the smaller size and the inability to provide directions to the donor/patient. Repeated removal and reinsertion to “check progress” is not desirable, as this could increase stress, especially in premature infants in neonatal care.

Thus, there is a need for a saliva sample collection apparatus which is designed for the special needs of pediatric and neonatal patients, sized for pediatric and neonatal patients, is safe for use, and optimized for the smaller saliva volumes produced by such patients.

### SUMMARY AND ADVANTAGES

A pediatric saliva sample collector includes a handle, an absorbent pad partially contained within the handle, a pad enclosure partially enclosing the portion of the absorbent pad extending outward from the handle, a mouth shield, and a positive capture retaining member extending through the absorbent pad to prevent detachment during use. The retaining member may also serve as a sample volume adequacy indicator. A docking station may be provided, the docking station having a cavity to receive the sample collector and an outlet port to direct expressed liquid sample into a collector.

A pediatric saliva sample collection system includes a pacifier portion having a receiver portion, a mouth shield, and a nipple portion extending from the receiver portion, the receiver portion and nipple portion each having an internal cavity in fluid communication with each other and adapted to receive a collector absorbent pad. A nipple portion includes a plurality of apertures providing fluid communication from a subject's mouth and tongue with the collector absorbent pad. The system may include a stiffener insertable into the pacifier receiver portion, the stiffener having an internal ridge to abut the second end of the collector handle when the collector is inserted into the pacifier. A collector portion includes a handle extending from a first end to a second end, and having a grip portion proximate the handle first end and a receiving cavity proximate the second end to receive a portion of an absorbent pad therein, and an absorbent pad insertable into the collector handle receiving cavity and extending outward therefrom. The absorbent pad may be cylindrical in cross section. A pad compression tube is provided to receive an absorbent pad and handle to express liquid sample therefrom.

The pediatric saliva sample collection system of the present invention presents numerous advantages, including: (1) ability to obtain saliva samples from infants, including premature infants; (2) ability to obtain saliva samples from infants having adequate sample volume to express liquid sample; (3) ability to obtain body fluid samples from infants for diagnostic and research uses without need to draw blood or other body fluids which cause pain and/or elevated stress levels for infants; (4) ability to utilize a standardized collector in combination with pacifiers of different size/dimensions for different size/age ranges of infants; (5) ability to efficiently obtain saliva samples from animals.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

FIG. 1 shows a perspective side view of a first embodiment.

FIG. 2 shows a close up side view of a portion of a first embodiment, with the receiver portion of the shield not shown in order to view the internals.

FIG. 3 shows a close up perspective side view of a portion of a first embodiment, with the receiver portion of the shield not shown in order to view the internals.

FIG. 4 shows a side view of the handle and shield portions of a first embodiment.

FIG. 5 shows a perspective view of the handle and shield portions of a first embodiment.

FIG. 6 shows a perspective view of an assembled first embodiment.

FIG. 7 shows a perspective side view of a second embodiment.

FIG. 8 shows a perspective side view of a second embodiment (opposite side from FIG. 7).

FIG. 9 shows side view of an absorbent pad and pad enclosure of a second embodiment.

FIG. 10 shows a another side view of an absorbent pad and pad enclosure of a second embodiment.

FIG. 11 shows a top perspective view of a docking station.

FIG. 12 shows a bottom perspective view of a docking station.

FIG. 13 shows a side perspective view of a docking station.

FIG. 14 shows a top perspective view of a docking station.

FIG. 15 shows a partial exploded view of a third embodiment, with a pacifier.

FIG. 16 shows a second partial exploded view of a third embodiment, with a pacifier.

FIG. 17 shows a partial exploded view of a third embodiment, with a pad compression tube.

FIG. 18 shows a side view of a pacifier of a third embodiment.

FIG. 19 shows a cutaway side view of a pacifier of a third embodiment.

FIG. 20 shows a second cutaway side view of a pacifier of a third embodiment, rotated 90 degrees from FIG. 19.

FIG. 21 shows a side view of a stiffener of a third embodiment.

FIG. 22 shows a cutaway side view of a stiffener of a third embodiment.

FIG. 23 shows a side view of a collector portion of a third embodiment.

FIG. 24 shows a side view of a pad compression tube of a third embodiment.

### REFERENCE NUMBERS USED IN DRAWINGS

Turning now descriptively to the drawings, in which similar reference characters denote similar elements throughout the several views, the figures illustrate the present invention. With regard to the reference numerals used, the following numbering is used throughout the various drawing figures:

| Reference | |
|---|---|
| Number | Description |
| 10 | First Embodiment |
| 12 | Handle |
| 14 | Absorbent Pad |
| 16 | Pad Enclosure |
| 18 | Shield |
| 20 | Retaining Member |
| 22 | Shield Receiver Portion |
| 24 | Shield Flange |
| 26 | Shield Front Side |
| 28 | Shield Backside |
| 30 | Pad Cavity |
| 32 | Pad Cavity Back Wall |
| 34 | Pad Cavity Sidewall |
| 36 | Pad Cavity Opening |
| 38 | Receiver Portion First Retention Cavity |
| 40 | Receiver Portion Second Retention Cavity |
| 42 | First Retainer Element Aperture |
| 44 | Second Retainer Element Aperture |
| 46 | Retaining Member First End |
| 48 | Retaining Member Second End |
| 50 | Pad Enclosure First Sidewall |
| 52 | Pad Enclosure Second Sidewall |
| 54 | Pad Enclosure First Sidewall First End |
| 56 | Pad Enclosure Second Sidewall First End |
| 58 | Pad Enclosure First Sidewall Second End |
| 60 | Pad Enclosure Second Sidewall Second End |
| 62 | Pad Enclosure First Sidewall Second End Aperture |
| 64 | Pad Enclosure Second Sidewall Second End Aperture |
| 66 | Absorbent Pad First End |
| 68 | Absorbent Pad Second End |
| 70 | Absorbent Pad Aperture |
| 72 | Absorbent Pad/Pad Enclosure Gap |
| 74 | Handle Ribs |
| 76 | Handle Grooves |
| 78 | Pad Enclosure End Wall |
| 110 | Second Embodiment |
| 112 | Handle |
| 114 | Absorbent Pad |
| 116 | Pad Enclosure |
| 118 | Shield |
| 120 | Retaining Member |
| 122 | Shield Receiver Portion |
| 126 | Shield Front Surface |
| 138 | First Retention Cavity |
| 140 | Second Retention Cavity |
| 150 | Pad Enclosure First Sidewall |
| 152 | Pad Enclosure Second Sidewall |
| 154 | Pad Enclosure First Sidewall First End |
| 156 | Pad Enclosure Second Sidewall First End |
| 158 | Pad Enclosure First Sidewall Second End |
| 160 | Pad Enclosure Second Sidewall Second End |
| 162 | Pad Enclosure First Sidewall Second End Aperture |
| 164 | Pad Enclosure Second Sidewall Second End Aperture |
| 166 | Absorbent Pad First End |
| 178 | Pad Enclosure End Wall |
| 180 | Pad Enclosure Nipple Portion |
| 182 | Pad Enclosure First Sidewall Shoulder |
| 184 | Pad Enclosure Second Sidewall Shoulder |
| 186 | Abutting Surface |
| 188 | Abutting Surface |
| 190 | First Crease |
| 192 | Second Crease |
| 200 | Docking Station |
| 202 | Docking Station Receiver |
| 204 | First Leg |
| 206 | Second Leg |
| 208 | Receiver First Cavity |
| 210 | Receiver Second Cavity |
| 212 | Receiver Outlet Port |
| 214 | Receiver First Cavity Bottom Wall |
| 216 | Receiver First Cavity Sidewall |
| 218 | Receiver First Cavity Bottom Apertures |
| 220 | Receiver Second Cavity Bottom Wall |
| 222 | [not used] |
| 224 | Receiver Second Cavity First Lateral Aperture |
| 226 | Receiver Second Cavity Second Lateral Aperture |
| 310 | Third Embodiment |
| 312 | Pacifier Portion |
| 314 | Stiffener |
| 316 | Removable Collector |
| 318 | Pad Compression Tube |
| 320 | Pacifier Receiver Portion |
| 322 | Nipple Portion |
| 324 | Mouth Shield |
| 326 | Pacifier Receiver Interior Cavity |
| 328 | Pacifier Receiver Interior Cavity First End |
| 330 | Pacifier Receiver Interior Cavity Second End |
| 334 | Pacifier Receiver Channel |
| 336 | Pacifier Receiver Interior Surface |
| 338 | Stiffener Exterior Ridge |
| 340 | Nipple Portion Base |
| 342 | Nipple Portion Terminal End |
| 344 | Nipple Portion Interior Cavity |
| 346 | Nipple Portion Interior Cavity First End |
| 348 | Nipple Portion Interior Cavity Second End |
| 350 | Absorbent Pad |
| 352 | Nipple Portion Exterior Surface |
| 356 | Stiffener First End |
| 358 | Stiffener Second End |
| 360 | Stiffener Interior Channel |
| 362 | Stiffener Exterior Surface |
| 364 | Stiffener Interior Ridge |
| 366 | Collector Portion Handle |
| 368 | Handle First End |
| 370 | Handle Second End |
| 372 | Collector Handle Second End Receiving Cavity |
| 374 | Collector Handle Second End Receiving Cavity Open End |
| 376 | Collector Handle Second End Receiving Cavity Enclosed End |
| 378 | Collector Seal |
| 380 | Stiffener Interior Channel Surface |
| 382 | Pad Compression Tube First End |
| 384 | Pad Compression Tube Second End |
| 386 | Pad Compression Tube Interior Shoulder |
| 388 | Pad Receiving Volume |
| 390 | Pad Compression Tube Outlet Port |
| 392 | Nipple Portion Apertures |
| 394 | Collector Handle Grip Portion |
| 396 | Collector Handle Thumb Pad |

### DETAILED DESCRIPTION

Before beginning a detailed description of the subject invention, mention of the following is in order. When appropriate, like reference materials and characters are used to designate identical, corresponding, or similar components in differing figure drawings. The figure drawings associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

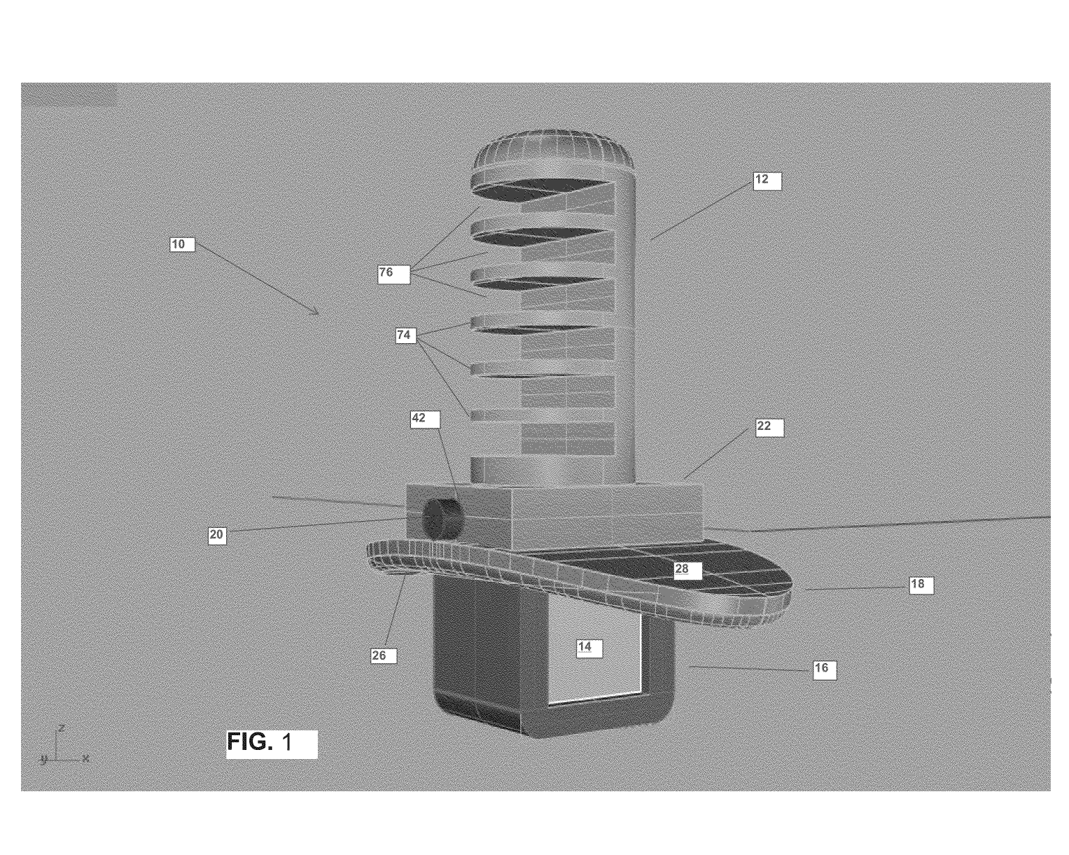

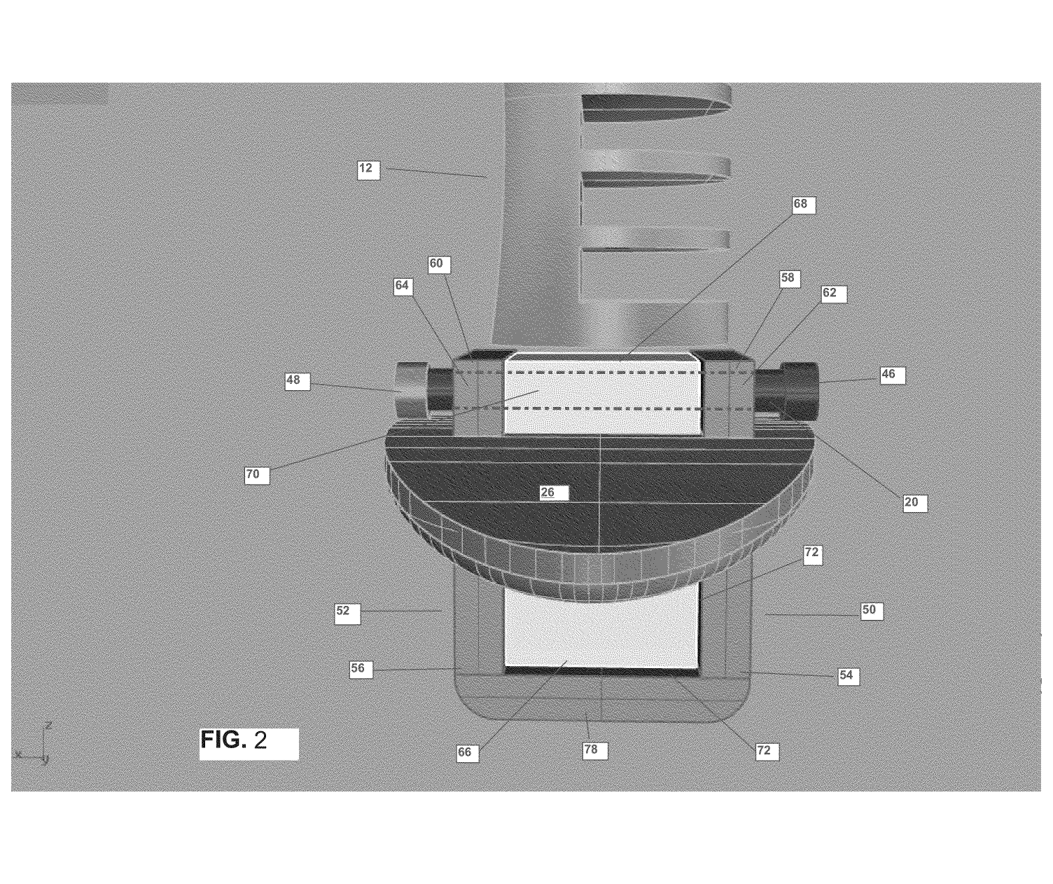

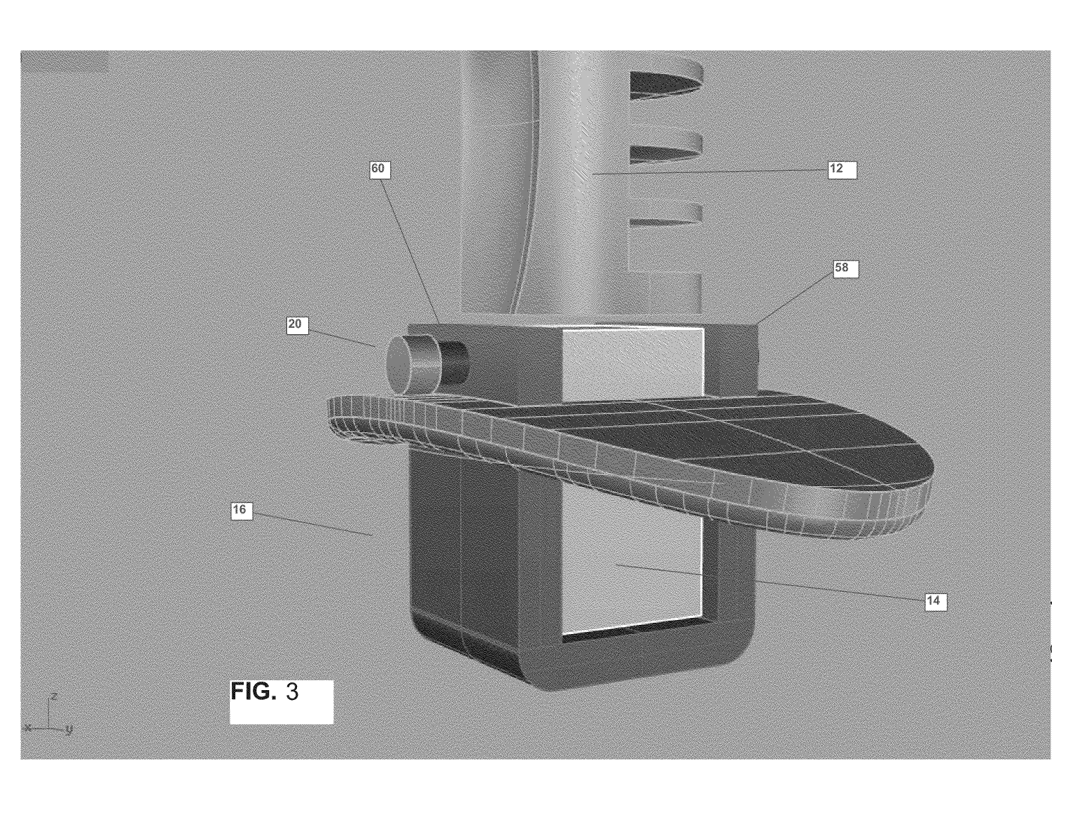

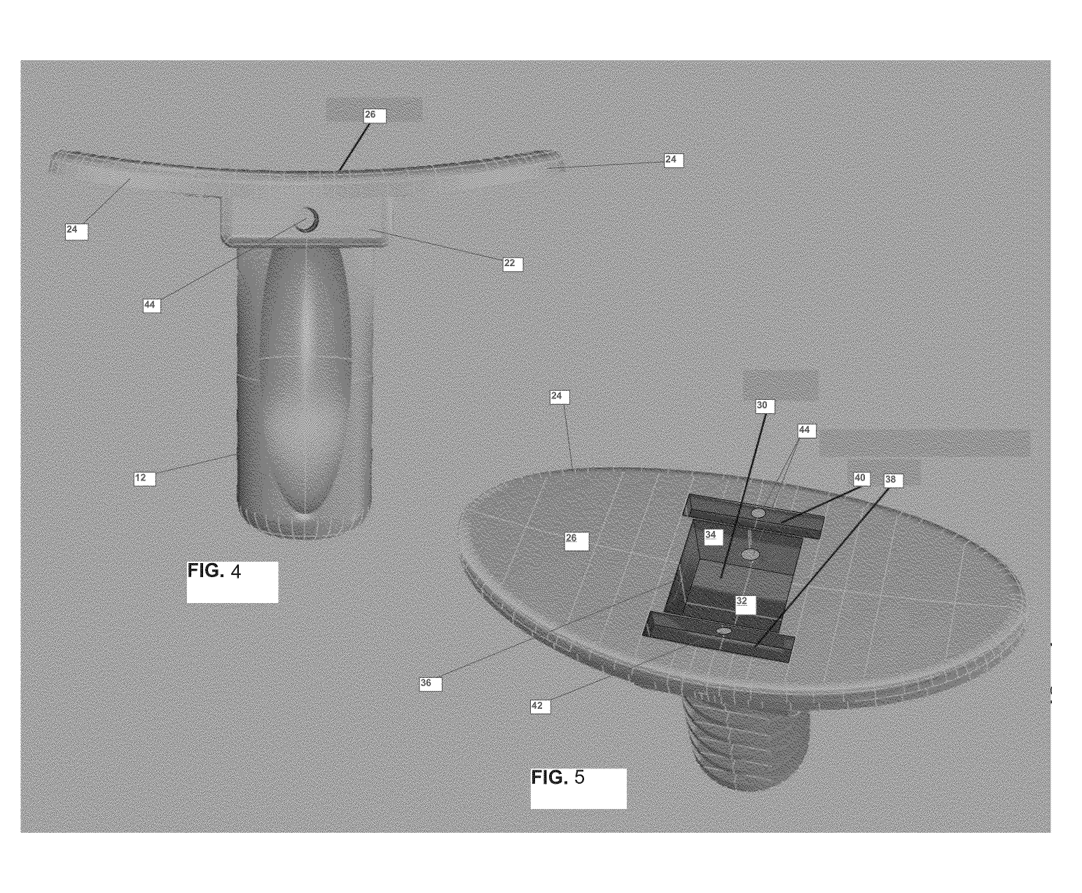

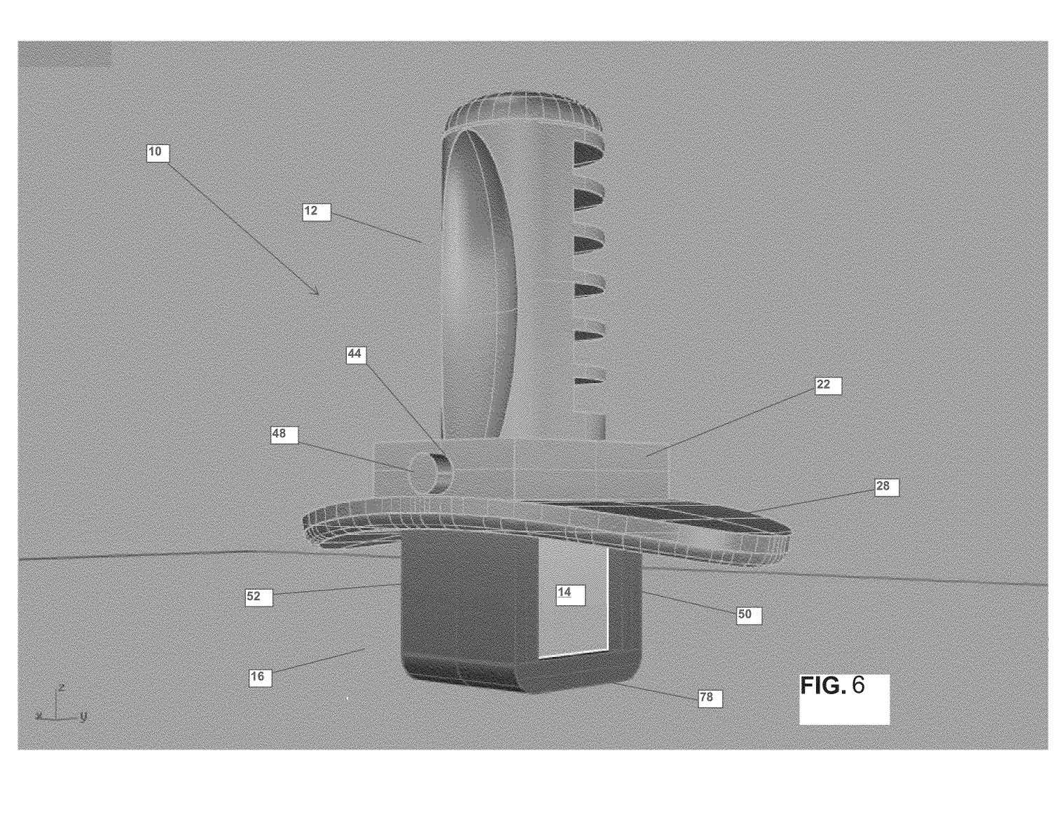

Referring to FIGS. 1-6, a first embodiment of a pediatric saliva sample collector 10 includes a handle 12, an absorbent pad 14, a pad enclosure 16, a shield 18, and a positive capture retaining member 20. In the embodiment, retaining member 20 also serves as a sample volume adequacy indicator (“SVAI”).

Shield 18 includes a receiver portion 22, and flange 24 extending laterally from receiver portion 22. Flange 24 includes a front side 26 and opposed back side 28. Receiver portion 22 provides a connection region for handle 12, absorbent pad 14, pad enclosure 16 and retaining member 20. Receiver portion 22 projects out from shield back side 28. Handle 12 connects rigidly to receiver portion 22. Receiver portion 22 includes pad cavity 30 defined by a liquid-tight back wall 32, a liquid-tight continuous sidewall 34, and cavity opening 36 which extends through front side 26 of shield 18. Receiver portion 22 further includes first and second retention cavities 38, 40, respectively, disposed on opposed sides of pad cavity 30. Retention cavities 38, 40 extend into receiver portion 22 are open to the front side 26 of shield 18.

In the embodiment, retaining member 20 acts as a volume indicator and a positive capture retaining mechanism. Retaining member 20 extends from a first end 46 to opposed second end 48, the retaining member 20 mounted transversely through receiver portion 22. When installed in saliva collector 10, retaining member 20 extends transversely through receiver portion 22 first retainer element aperture 42, through first retention cavity 38, through pad cavity 30 within absorbent pad aperture 70, through second retention cavity 40, and through opposed second retainer element aperture 44. First and second retention caps are installed at retention member first and second ends 46 and 48, respectively, to fix retention member 20 in place. In the embodiment, retaining member 20 comprises a light pipe.

In the embodiment, pad enclosure 16 comprises a generally U-shaped retainer having an end wall 78 and opposed parallel first and second sidewalls 50, 52, extending from respective first ends 54, 56, joined to end wall 78, to respective second ends 58, 60. First and second sidewall second ends 58, 60, respectively, couple to shield receiver portion 22 by inserting into first and second retention cavities 38, 40, respectively. Pad enclosure sidewall second ends 58, 60, each include an aperture 62, 64, respectively, proximate their terminal ends, to receive retainer element 20 there through. Pad enclosure 16 is made from soft medical-grade silicone rubber to permit compression of absorbent pad 14 to express sample, and provide comfort to the user.

Absorbent pad 14 is shaped to fit within pad enclosure 16 without significant gaps when it is saturated, in order to maximize sample collection volume and efficiency of expression. Therefore, when dry, there is a small gap 72 between the outer surfaces of absorbent pad 14 and pad enclosure 16, but when saturated with liquid sample absorbent pad 14 expands slightly to be in contact with pad enclosure 16 without substantial compression. In the embodiment, absorbent pad 16 is a rectangular block extending from a first end 66 to a second end 68 and having aperture 70 extending transversely there through proximate second end 68 to receive retaining member/SVAI 20.

In the embodiment, the portion of absorbent pad 14 extending beyond shield 18 is approximately a 1 cm (0.4 in) cube, in order to be small enough to fit the mouth of a premature newborn. The dimensions may be varied to accommodate infants of different age ranges.

Handle 12 is provided with alternating ribs 74 and grooves 76 extending transversely and distributed along most of the length of handle 12, providing comfortable and positive grip in wet conditions.

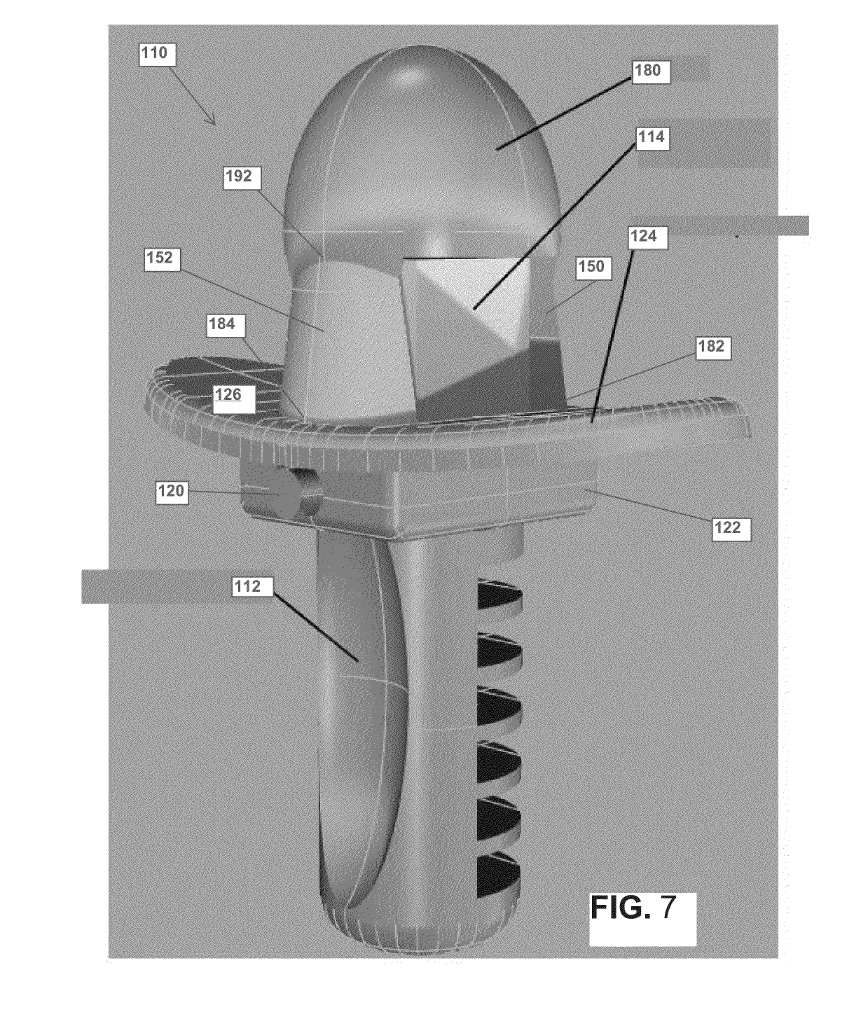

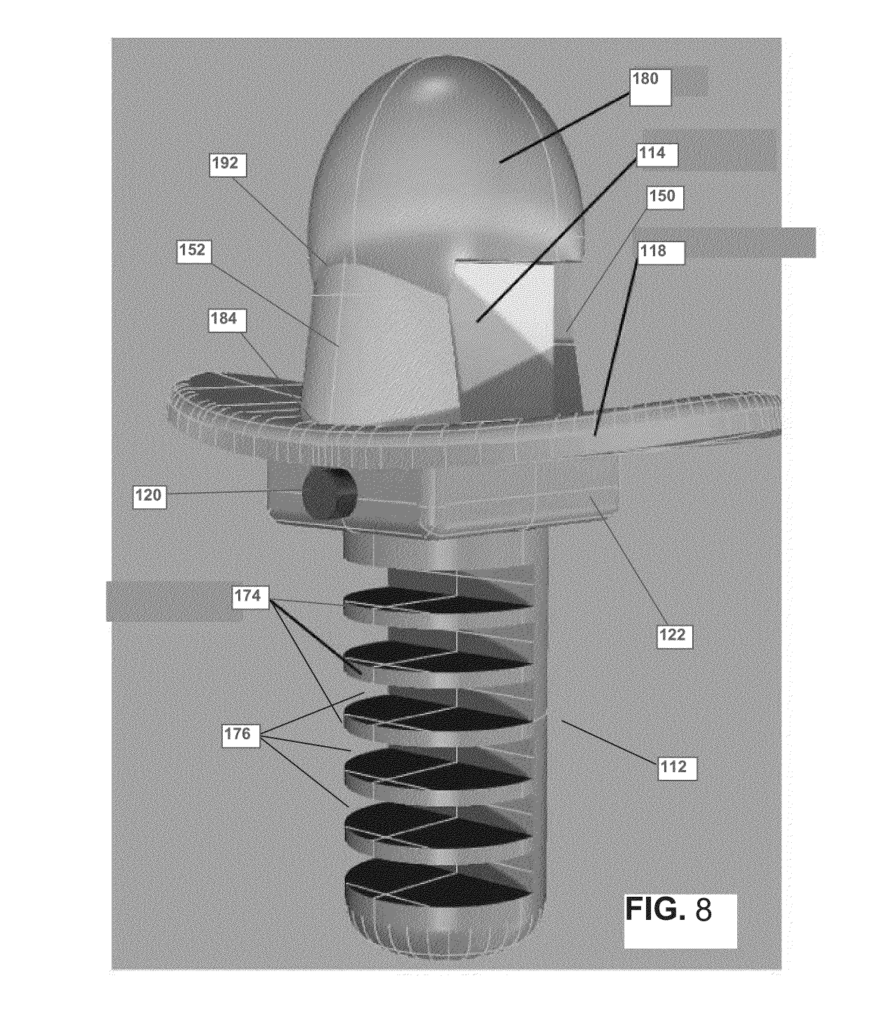

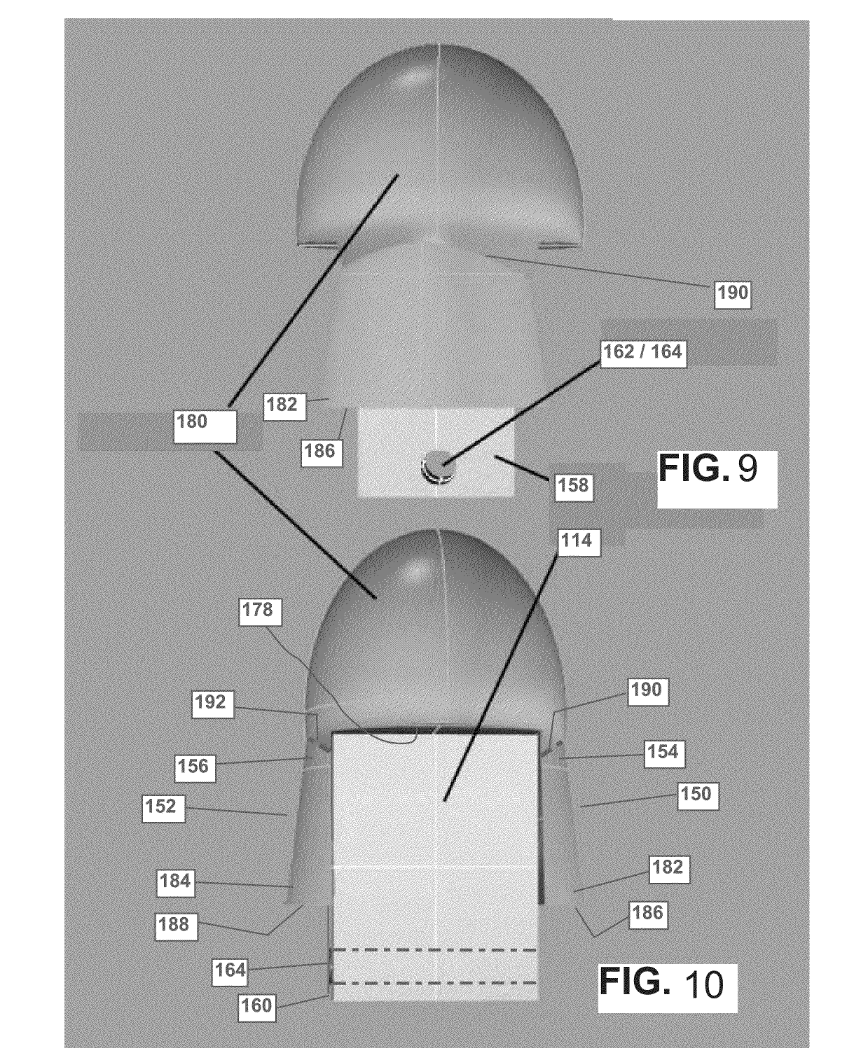

Referring to FIGS. 7-10, a second embodiment 110 is shown. The second embodiment includes a handle 112, an absorbent pad 114, a pad enclosure 116, a shield 118, and a positive capture retaining member 120, which are similar to the corresponding structures of the first embodiment 10. In the embodiment, retaining member 120 also serves as a sample volume adequacy indicator (“SVAI”).

In the second embodiment, pad enclosure 116 incorporates a nipple structure to improve comfort for infants and toddlers, and encourage sucking action which will increase saliva production and ensure pad saturation. Pad enclosure 116 comprises a generally U-shaped retainer having an end wall 178 and opposed parallel first and second sidewalls 150, 152, extending from respective first ends 154, 156, joined to end wall 178, to respective second ends 158, 160. First and second sidewall second ends 158, 160, respectively, couple to shield receiver portion 122 by inserting into first and second retention cavities 138, 140, respectively (not shown). Pad enclosure sidewall second ends 158, 160, each include an aperture 162, 164, respectively, proximate their terminal ends, to receive retainer element 120 there through.

Pad enclosure 116 includes nipple portion 180 extending beyond end wall 178. Pad enclosure first and second sidewalls, 150 and 152 include flared shoulders 182, 184, respectively, forming abutting surfaces 186 and 188, respectively, which go against shield front surface 126. Flared shoulders 182, 184, taper from proximate abutting surfaces 186, 188, to locations proximate first and second sidewall ends 154, 156, respectively, forming first and second creases 190 and 192, respectively. In this way, flared shoulders 182, 184, provide stability and support for pad enclosure 116, allow sidewalls 150, 152 to be placed in slight tension when retention element 120 is inserted, but creases 190, 192, ensure pad enclosure 116 is compressible to express sample.

Nipple portion 180 may be formed with an interior cavity in communication with the cavity formed by sidewalls 150, 152, to receive absorbent pad first end 166 therein, pernmitting use of a larger absorbent pad 114 within the same overall dimensions.

The overall shapes of each of the first and second embodiments also comprise novel and nonobvious aesthetic designs, which are separable from the functional features.

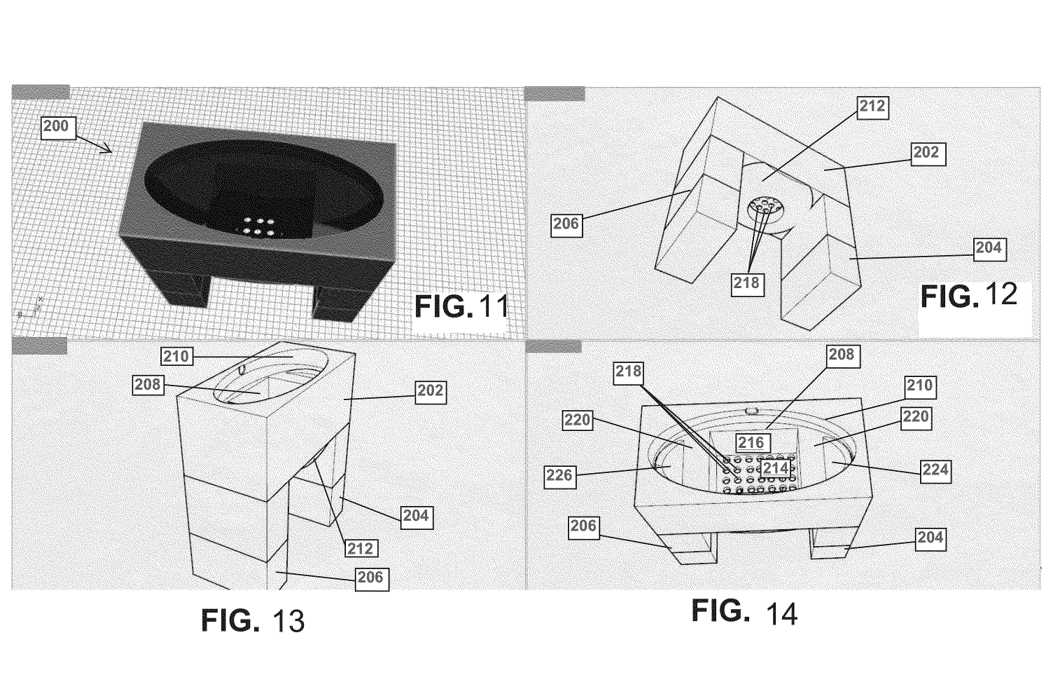

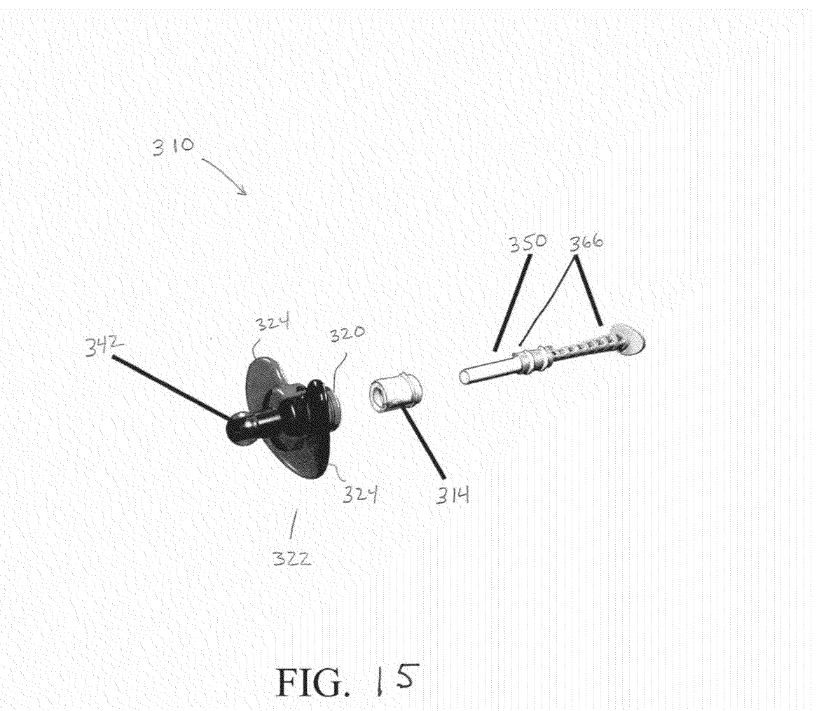

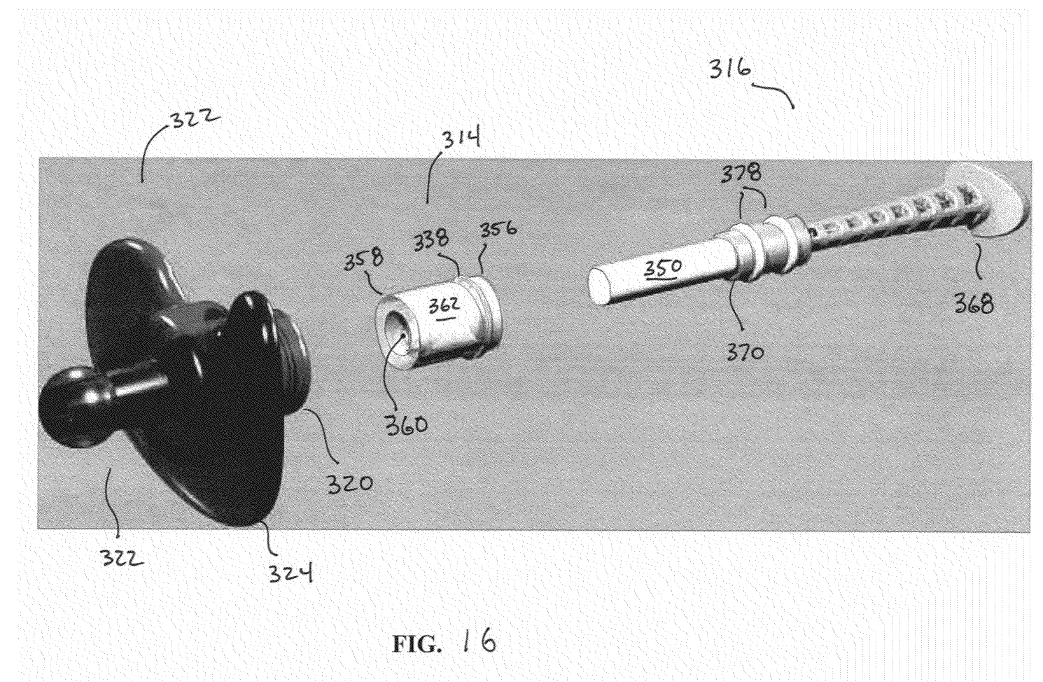

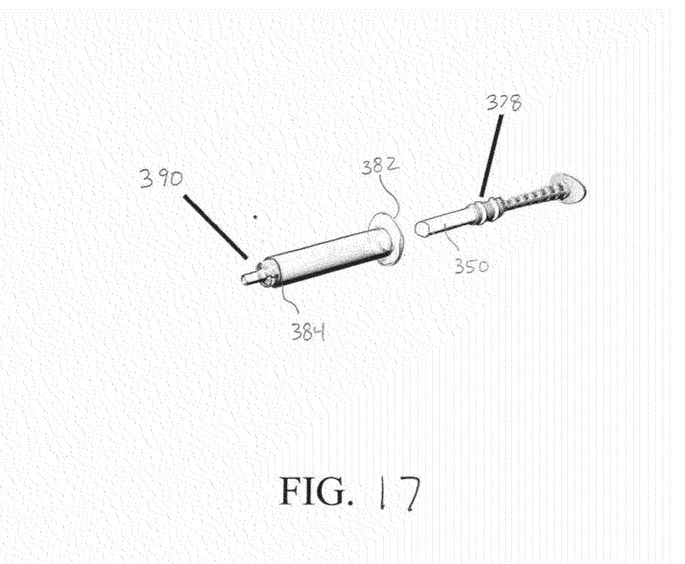

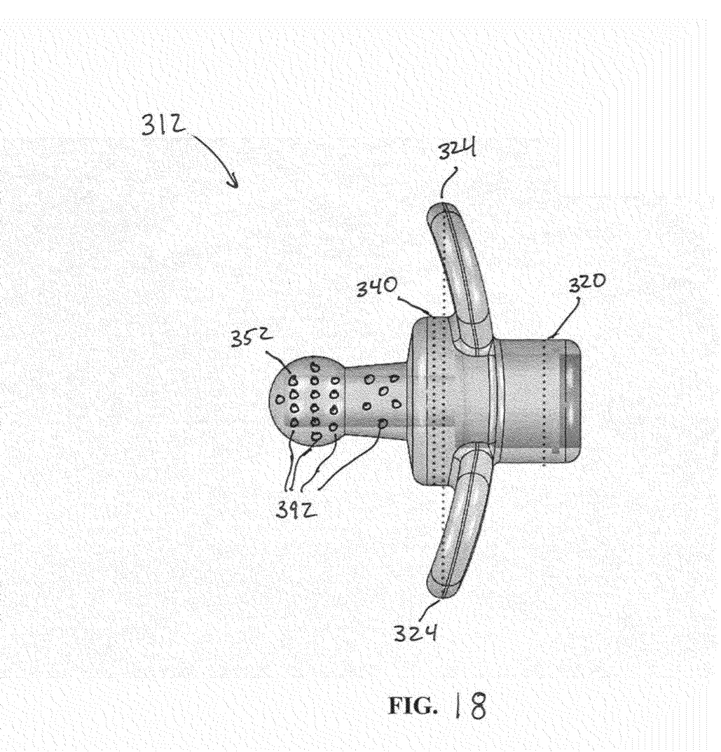

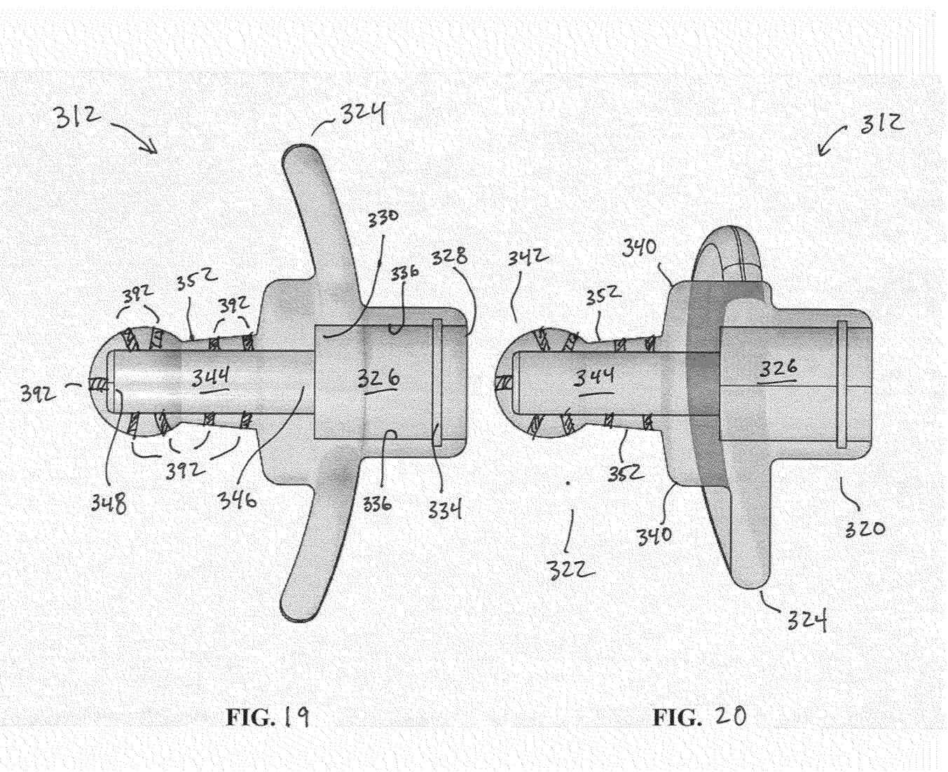

Referring to FIGS. 11-14, a docking station 200 is shown, for use with a pediatric saliva sample collector 10 or 110. Docking station 200 includes a receiver 202 and first and second legs 204, 206, respectively, extending from the underside of receiver 202. Receiver 202 includes first and second cavities 208, 210, respectively, in fluid communication with each other and an outlet port 212 disposed on the underside of receiver 202, which are shaped to receive and compress a pediatric saliva collection device, for example the first and/or second embodiments 10, 110. First cavity 208 is defined by a bottom wall 214 and enclosing sidewall 216. First cavity bottom wall 214 includes a plurality of apertures 218 providing fluid communication between first cavity 208 and outlet port 212. Sidewall 216 defines the depth of first cavity 208, which in the embodiment is approximately equal to the thickness of pad enclosure 16 and pad 14 extending out from shield 18 when fully compressed. Second cavity 210 includes a bottom wall 220 providing a shoulder for sample collector flange 24 to go against. In the embodiment, receiver 202 includes opposed first and second lateral apertures 224, 226, respectively, in fluid communication with second cavity 210 and outlet port 212, to port overflow liquid sample into outlet port 212. Outlet port 212 is configured to couple to standard Eppendorf collection tubes. Outlet port 212 may incorporate a tapered or funnel receiver to receive discharge from apertures 218 and lateral apertures 224, 226, for porting to a separable collection tube, such as a sealable Eppendorf tube.

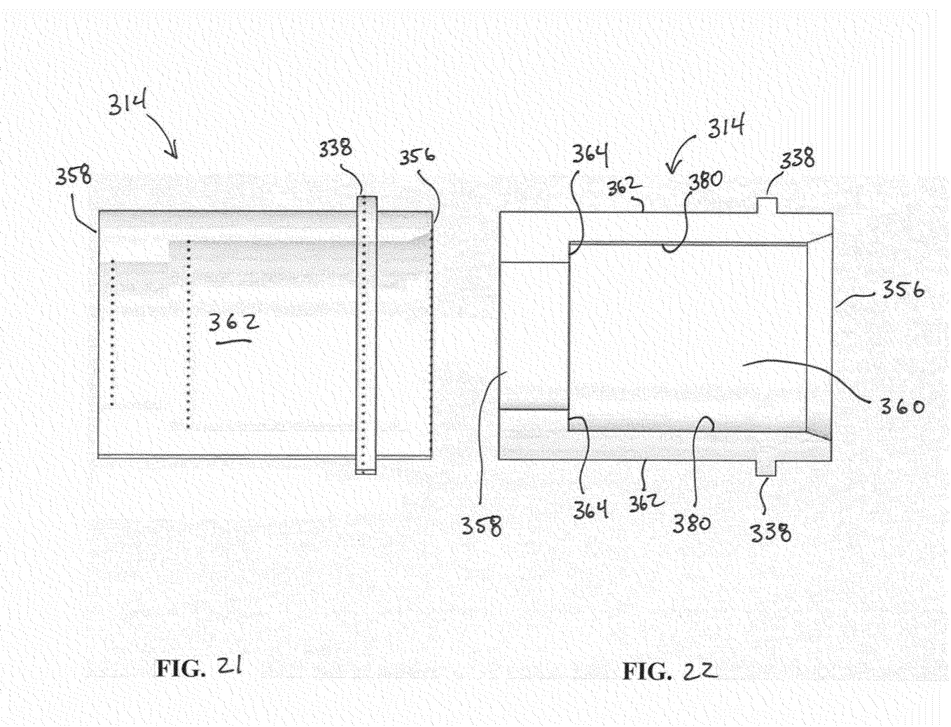

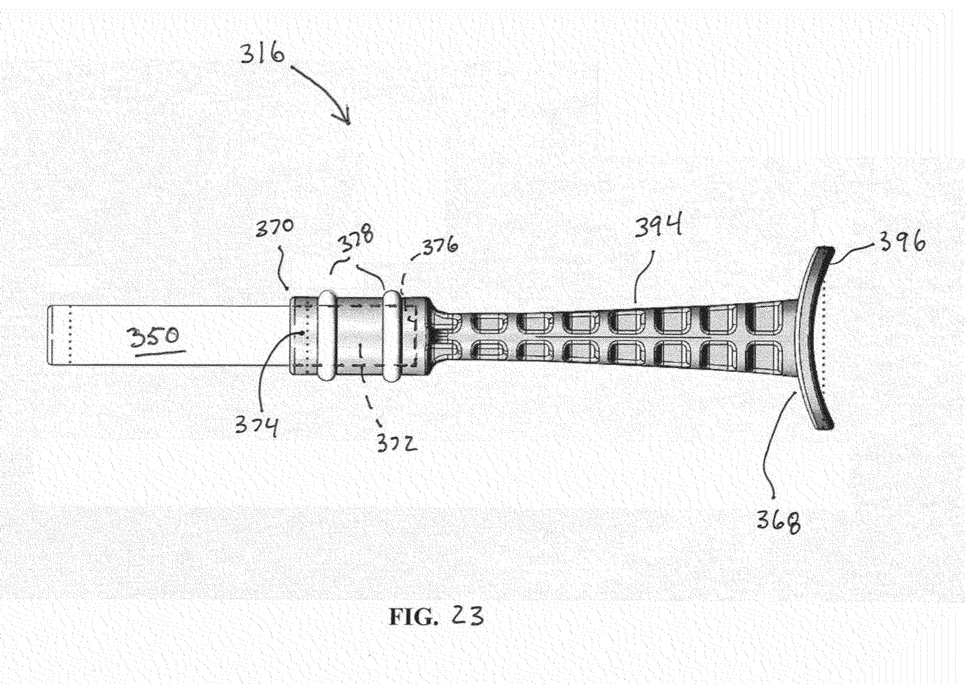

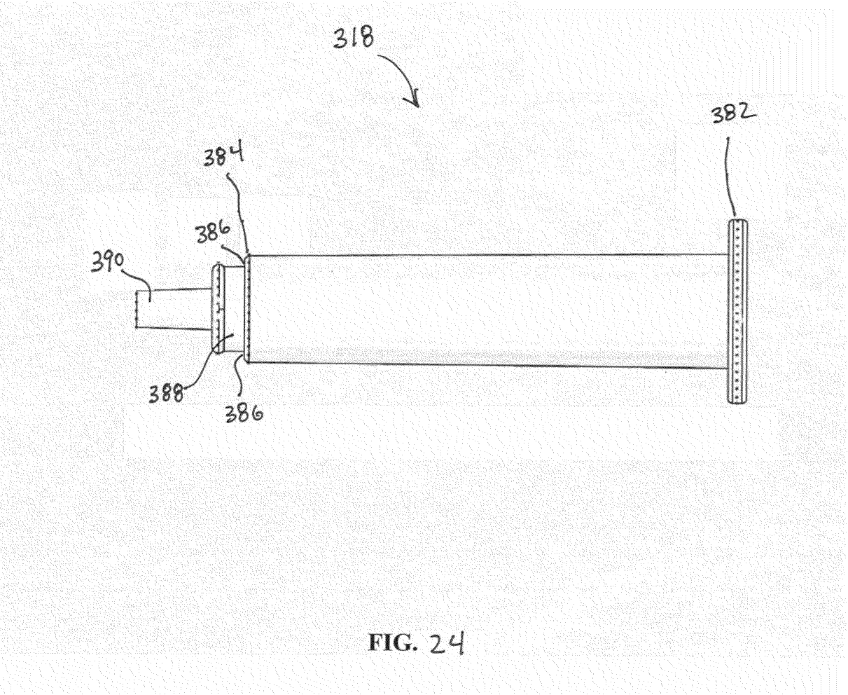

Referring to FIGS. 15-24, a third embodiment 310 is shown. A third embodiment 310 includes a pacifier portion 312, a stiffener 314, a removable collector 316 and a pad compression tube 318 insertable over removable collector 316.

Pacifier portion 312 includes receiver portion 320, nipple portion 322, and mouth shield 324. Receiver 320 includes an interior cavity 326 extending from a first open end 328 to a second end 330. Receiver interior cavity 326 is in fluid communication with nipple interior cavity 344. In the embodiment, receiver interior cavity 326 includes channel 334 circumscribing the interior surface 336, channel 334 disposed to engage corresponding ridge 338 circumscribing the exterior surface 362 of stiffener 314, thereby more firmly retaining stiffener 314 in place. In the embodiment, receiver interior cavity 326 is dimensioned to receive stiffener 314 snugly therein.

Nipple portion 322 has exterior dimensions selected to accommodate a child within a selected size or development range. Nipple portion 322 projects from a base portion 340 proximate receiver portion 320 to a terminal end 342. Nipple portion 322 includes an interior cavity 344 extending from a first open end 346 proximate receiver interior cavity second end 330, and in fluid communication therewith, to a second end 348 proximate nipple portion terminal end 342. Nipple interior cavity 344 is dimensioned to receive the selected collector absorbent pad 350 therein. Nipple portion 322 includes a plurality of apertures 392 extending from exterior surface 352 to nipple interior cavity 344, providing fluid communication between the subject's interior mouth and tongue to absorbent pad 350.

Mouth shield 324 comprises a flange extending laterally outward from a region proximate the connection of nipple portion base 340 and pacifier receiver portion 320. In the embodiment, pacifier portion 312 is made from medical grade silicone rubber or similar soft autoclavable material.

Stiffener 314 is insertable into pacifier receiver interior cavity 326. Stiffener 314 generally comprises a short stub tube extending from a first open end 356 to a second open end 358, defining a stiffener interior channel 360. Stiffener 314 includes exterior ridge 338 circumscribing the exterior surface 362 of stiffener 314, and disposed to engage channel 334 when stiffener 314 is inserted into pacifier receiver 320. Stiffener interior channel 360 includes interior ridge 364 disposed proximate second open end 358, extending inward sufficiently to abut collector handle second end 370 when collector absorbent pad 350 is inserted into nipple interior cavity 344. In the embodiment, interior ridge 364 defines the interior cross section of stiffener second open end 358, and is approximately equal to the cross section of absorbent pad 350. In the embodiment, stiffener 314 is made from a semi-rigid medical-grade autoclavable plastic.

Collector portion 316 includes handle 366 extending from a first end 368 to a second end 370. Collector portion 316 includes receiving cavity 372 disposed proximate handle second end 370 and extending from an open end 374 to an enclosed end 376, and dimensioned to receive an end portion of an absorbent pad 350. Collector portion 316 further includes an absorbent pad 350 partially contained within collector receiving cavity 372 and extending therefrom. In the embodiment, seal 378 includes a pair of O-rings to seal against the interior surface 380 of stiffener 314. In the embodiment, handle second end 370 engages against stiffener interior ridge 364 when inserted, to ensure absorbent pad 350 is not compressed when inserted into nipple interior cavity 344. Collector portion handle 366 includes a grip portion 394 and a thumb pad 396 proximate handle first end 368.

Pad compression tube 318 extends from a first open end 382 to a second end 384, the first end 382 dimensioned to fit over the handle second end 370 and seal against O-rings 378 during compression of absorbent pad 350 to express liquid sample. Pad compression tube 318 further includes an interior shoulder 386 to abut collector handle second end 370 at full compression, a pad receiving volume 388 to contain the compressed absorbent pad 350 and maintain alignment, and outlet port 390 in fluid communication with the pad compression tube 318 interior volume and the pad receiving volume 388. In the embodiment, outlet port 390 is removably connectable to a selected type of sample collection tube, such as an Eppendorf tube.

In operation, a first embodiment is demonstrated, referring to FIGS. 1-6 and 10-14. An apparatus 10 is assembled by inserting absorbent pad 14 into pad cavity 30 within receiver 22 having handle 12 fixed thereto. Pad enclosure 16 is mounted over pad 14, with first and second sidewall ends 58 and 60 inserted into first and second retention cavities 38, 40, respectively. Retaining element 20 is inserted through the assembly and secured in place with end caps secured at retaining element first and second ends 46 and 48. The user places the absorbent pad/pad enclosure into their mouth for a time, at which point the sample volume adequacy indicator (in the embodiments, retaining element 20) is viewed to verify adequate volume is retained in absorbent pad 14.

The apparatus 10 is removed and inserted into docking station 200, compressed, and the expressed sample discharged through outlet port 212 retained for testing. Apparatus 10 may be retained for further testing of biological materials retained by absorbent pad 14 after expression.

Referring to FIGS. 15-24, the operation of a third embodiment 310 is demonstrated. The embodiment 310 may be provided as an assembled apparatus ready for use, or as a system, to be assembled at point-of-use, wherein multiple pacifiers 312 are provided having different size nipple portions 322, the appropriate size to be selected by the user based on the child's size and physical condition. In this way, a common collector portion 316 can be used with various pacifier nipple sizes.

Similarly, multiple sizes and types of absorbent pads 350 may be provided, the appropriate size/type to be selected by the user based on the child's size and physical condition, as well as the test regimen to be conducted on the saliva sample.

In use, a stiffener 314 is inserted into the selected pacifier receiver 320 until ridge 338 engages channel 334. A removable collector 316 having the selected absorbent pad 350 inserted into receiving handle second end cavity 372 is inserted into the pacifier 312 until handle second end 370 abuts stiffener interior ridge 364 and absorbent pad 350 extends into nipple cavity 344. The nipple portion 322 is then placed into the subject's mouth, which stimulates sucking/masticating and saliva production, which saturates absorbent pad 350 by flowing through apertures 392. The apparatus 310 is removed from the subject, the collector handle 366 and absorbent pad 350 are removed from the pacifier 312, inserted into the pad compression tube 318, and the absorbent pad 350 compressed into pad receiving volume 388 and liquid sample expressed through outlet port 390.

Those skilled in the art will recognize that numerous modifications and changes may be made to the preferred embodiment without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the preferred embodiment is essential. Other embodiments are possible, their specific designs depending upon the particular application. As such, the scope of the invention should not be limited by the particular embodiments herein described but should be defined only by the appended claims and equivalents thereof.

It is understood that the optimum exterior dimensions of the nipple will vary in size, depending on the size of the infant or child subject. In practice, a series of nipples may be provided, each having dimensions selected as a median for given size range of subjects.

In addition to human infants, the apparatus described and claimed herein are useful and applicable to obtaining saliva samples from animals by selecting an appropriately dimensioned pacifier/nipple, as the enclosing nipple stimulates sucking/mastication and protects the absorbent pad from the animal's chewing during sample collection. In this sense, “pediatric” implies directed to subject populations which are mentally or physically unable to adhere to procedures for conventional saliva sample collection apparatus.

We claim:

1. A pediatric saliva sample collection apparatus, comprising:
a handle extending from a first end to a second end;
a shield portion, the shield portion including a receiver portion and laterally extending flange, the shield receiver portion connected to the handle second, the receiver further including a pad cavity;
an absorbent pad having a first end disposed within the receiver portion pad cavity and a second end protruding out from the pad cavity;
a pad enclosure connected to the receiver portion and partially enclosing the protruding portion of the absorbent pad along opposed sides and the second end of the absorbent pad; and,
a positive capture retaining member extending coupling the pad enclosure and absorbent pad to the receiver portion.

2. The apparatus of claim 1, further comprising a docking station, the docking station including a receiving cavity and an outlet port in fluid communication with the receiving cavity.

3. A pediatric saliva sample collection apparatus, comprising:
a pacifier portion, a stiffener, a removable collector and a pad compression tube;
the pacifier portion comprising a receiver portion including a first interior cavity, a mouth shield extending laterally out from the receiver portion, and a nipple portion extending from a base connected to the receiver portion to a terminal end, the nipple portion further including an outer surface, a second interior cavity in fluid communication with the first interior cavity, and a plurality of apertures extending from the nipple outer surface to the second interior cavity;
the stiffener insertable into the pacifier receiver portion first cavity, the stiffener comprising a tube extending from a first end including a first opening to a second end including a second opening, the stiffener further comprising an interior shoulder;
the removable collector comprising a handle extending from a first end to a second end, the handle including a grip portion proximate the first end and a pad receiving cavity proximate the second end, the collector further comprising an absorbent pad partially insertable into the pad receiving cavity and protruding outward therefrom, and a seal disposed proximate the handle second end, the removable collector removably insertable into the pacifier portion with the seal sealingly engaged against the stiffener interior surface; and,
the pad compression tube extending from a first open end to a second end, the pad compression tube including an interior shoulder proximate the pad compression tube second end to engage the handle second end, a pad compression receiving cavity extending therefrom, and an outlet port in fluid communication with the pad compression tube interior, the pad compression tube dimensioned to receive the removable collector therein.
